(19) United States
(12) Patent Application Publication
(10) Pub. No.: US 2003/0028069 A1
(43) Pub. Date: Feb. 6, 2003

(54) Bio-enhanced magnetic device for promoting hair growth

(76) Inventor: Carmelita S. Santiago, San Diego, CA (US)

Correspondence Address: Don E. Erickson, Law Offices, PMB 182, 3830 Valley Center Drive # 705, San Diego, CA, 92130, US

(21) Appl. No.: 09/921,211

(22) Filed: Aug. 2, 2001

(51) Int. Cl. A61N 2/00

(52) U.S. Cl. 600/9

(57) ABSTRACT

A device and method for stimulating a beneficial blood circulation especially on the scalp area to prevent, stop and control hair loss, regenerate hair, as well as alleviate pain by means of strategically-placed magnetic bodies incorporated in a portable, grippable model of known geometric structures for generating a healing force of form energy. The magnetic bodies in form of permanent magnets generate an amplified and varying magnetic force in response to the modifying action of the form energy called shape wave. One or more permanent magnets are attached about the body of the grippable device secured by attachment means; at least one permanent magnet is located at the base portion, and another permanent magnet may be located around the wall portions or in designated places within and or outside the grippable device. The magnets are positions such that their pole surfaces face outwardly to contact two separate body parts at the same time so as to deliver half waveform each of positive and negative magnetism. When such pole faces are aligned with the body's own polarity centers and then massaged lightly by the user's own gripping hand using the effleurage type of massage, a more usable and more penetrating magnetic field is extended to access the body's own polarity centers and make a complete circuit thereby eliminating the possibility of polarity imbalance through improper and indiscriminate use of magnetic therapy device.

### FIELD OF THE INVENTION

The present invention relates in general to a therapeutic device and method of controlling and stopping falling hair and promoting hair growth naturally based on magnetism and more specifically to a magnetic device that boosts the ordinary strength of magnetic flux by means of synergistic combination of magnetic field and the form energy generated by known special, specific geometrical forms, such as the pyramid shape.

### BACKGROUND OF THE INVENTION

Alopecia, the lack or loss of hair, in its many forms is the continuing scourge of modern day living because of its downgrading effect, not only on the persona but more so on the psyche and self-image of the sufferer. It has become increasingly important in recent years to develop a product and/or procedures that will effectively inhibit or prevent the signs of baldness.

Prior attempts to eliminate the effects of baldness involved a variety of products and/or procedures, including massages and massaging compounds, shampoos, cream and oils, wigs and hairpieces, pharmaceutical formulations, micro-current treatment and other electrical stimulation techniques, and, more drastically, the more invasive hair transplant and surgery procedures. The principal problem today with hair-promoting products and procedures is providing an acceptable balance between cost, effectiveness, safety, and ease of use or application. The fact that no single product has really captured the market is an indication that an acceptable hair promoting product and/or procedure is yet to appear.

As a result of the failure of allopathic or conventional medicine to effectively treat certain medical conditions such as those described above, there is a growing tendency to explore and refine the paths of alternative medicine. One of these alternative practices is bio-magnetic therapy, which has been widely known and well documented for its capacity to carry oxygen. For therapeutic purposes, magnetic fields are commonly used for reduction of pain and inflammation in tissues. Over the years magnetic field has been clinically found to increase blood flow, to change the migration of calcium ions to or from the bone, to influence the permeability of sodium and potassium ions through the cellular membrane, to favor or inhibit the liberation of small quantities of neurotransmitters, to affect the autonomous nervous system, to alter the pH balance of various body fluids, to alter hormone production from endocrine glands and to alter the enzymatic activity and other biochemical processes of the human body. Additionally, the application of magnetic fields to plants is likewise believed to have beneficial effects on plant germination and growth.

Another alternative path that is being practiced in many parts of the world and gaining acceptance in the United States is the utilization of form energy or shape wave which includes the pyramid energy therapy. It is utilized for the relief and cure of nervousness, insomnia, headache and related troubles. The pyramid therapy, besides being used for increasing ones mood and well-being, is also credited for its positive effect on plant life, where the growth is accelerated and the foliage and produce harvest is increased.

There are various and divergent factors that can contribute to hair loss but it is now widely accepted that the single most important factor is impaired circulatory and metabolic function which also affects the scalp. Most of the emphasis in alopecia research to find a cure for the problem has been placed on scalp stimulation as the leading player in the prevention and control of inordinate hair loss, elimination of the signs of baldness and promotion of hair growth.

Blood flow is the life giver. Its unimpeded circulation performs two important functions in the body: 1) to deliver the needed nutrients and substances to every cells and tissues and, 2) to absorb the impurities and toxins that must be excreted via the eliminatory channels. Poor blood circulation means these functions, along with the metabolic function, are critically compromised. Taking cognizance of this fact, the prior art has investigated in and disclosed other stimulative treatment of human tissues for various medical reasons, including afflictions of the head and the scalp, utilizing various means like, for example, through deep tissue massage and ionic therapy, as found in U.S. Pat. No. 5,843,005; through simple magnetic devices in U.S. Pat. No. 5,843,005, JP8117349A2 and JP10015084A2; through the use of vacuum, as found in U.S. Pat. No. 5,454,778 and JP 9299422; by means of electrodes with varying signals and frequency in U.S. Pat. Nos. 5,336,247 and 3,946,745; by means of electrode and modulated waveform in U.S. Pat. No. 3,872,859; through electrical signals of opposite polarity, as found in U.S. Pat. No. 3,946,745 and CN1252985A; through electro-acupuncture techniques such as that described in U.S. Pat. Nos. 4,556,064, 3,897,789, and 5,782,858; through magnetic field therapy in U.S. Pat. Nos. 4,727,857, 5,632,720, 5,116,304 and Swiss Pat. No. 313,679; or a combination of magnetism and sensory stimulation as found in JP2029253; and electromagnetic or combination of electrical and magnetic stimulation as found in U.S. Pat. Nos. 4,197,851, 4,744,350, 4,838,850, 4,850,959, 5,967,967, 6,042,531, 6,007,501 and JP1056063. The foregoing is a non-exhaustive list of prior art references known by those of ordinary skill in the art and subscribing to the importance of stimulative treatment in treating physical problems. Although some of them consider the beneficial effect of stimulating the head area, no specific and definitive mechanism is provided for stimulating repair of worsening hair nor any specific patterns of treatment or scalp stimulation technique is discussed.

Magnetic fields have also been used without electric fields to stimulate biological tissues., both constant and static, as found in U.S. Pat. Nos. 5,143,056, 5,389,981, and JP 342698A2, JP 217934A2, and dynamic, as found in U.S. Pat. No. 5,817,000, but in all cases the stimulation has not involved application of a static magnetic field to the head area of a subject for the purpose of scalp invigoration and stimulation. In most cases too, the combination of sensory stimulation is lacking.

Magnetic treatments have attained some degree of success in the matters of pain control, bone regeneration and brain treatment. The difficulty in the past has been with respect to providing an effective field, neither too strong nor too weak, particularly in various site to be effected for various and different ailment, so that a substantial proportion of the blood flowing through the skin area is subjected to a field of such suitable intensity, depth and frequency, and for a long enough period of time that the desired blood stimulation will be achieved.

Simple magnetic devices that come in different shapes and packaging and have the drawback of failing to achieve a sufficient therapeutic efficacy unless held affixed to the body for a prolonged period of time, because they are unable to give a great stimulus. Magnetic bands are well known, but they give indirect and temporary efficacy only. These are because static magnetism lacks the varying magnetic field necessary for optimization. It has therefore been desired to provide a magneto-therapeutic device for hair promotion and regeneration with a magnetic field that will act directly on the affected part of the body to achieve an optimized therapeutic effectiveness.

One of the problems encountered and viewed as an obstacle to success is the optimization of magnetic stimulation without the accompanying undesirable fringe effects caused by the use of electrical power. Furthermore, contrary to claims that magneto-therapy is harmless, it is believed that excessive and improper use of magnetic fields especially the single polarity type would harm the body. The traditional Chinese remedies even have herbal preparations and acupuncture points and techniques for those suffering from yin and yang imbalance caused and/or aggravated by excessive and improper magnetic stimulation. The shortcomings and defects of prior art, including their side effect and lack of significant treating result, have resulted in their not being widespread and popular especially in the area of scalp treatment for hair regeneration.

The prior art also has disclosed the application of the energizing power of the pyramidal configuration in solar energy collection and concentration, to wit, U.S. Pat. Nos. 4,509,501 5,829,427.

From superstitious status, utilization of pyramid energy has gained a respectable status due to results gained from exhaustive and long-standing research from empirical to scientific level to fathom its mystery. Its more popular usage is in the area of meditation, mind healing and consciousness, and higher awareness enhancement. Pyramidologists believe that the pyramid shape directs the electromagnetic and light energy from the stars surrounding the earth and other planets, hence it is thought to act as a tuner for the earth. Their belief was bolstered by the discovery that the pyramids at Gizah sit in perfect alignment with the three solar stars. Thus the Bauval Correlation Theory as to the motivation in the building of the Pyramid was formed when Bauval noticed that the positioning and detailed structure of the Gizah Pyramids are accurately related and are a mirror image to the positions of astronomical bodies in the constellation of Orion, specifically the Belt Stars of Orion.

There are some unique and beneficial qualities derived from the pyramid, as born out by years of investigation and word-wide experimentation on various levels and motivation. Pyramid replicas, besides the above-mentioned usage, produce preservation in organic matters, accelerate plant growth and increase plant yields, improve the flavor of charged foods and liquids and sharpen dull metals including blades, knives and even electric razors.

The energy or waveform emitted by reason of its pyramidal shape has been called the energy of form or shape wave. Other cultures call it prana, chi or bioplasmic energy. A Kirlian photograph of a pyramid model would also show radiating emanations, like bursts of energies, at the apex extending upward. The pyramid shape is one of certain geometric forms that have been known and accepted to accumulate, concentrate, direct, amplify and transform energy, the other forms being the conical and spherical forms.

The non-exhaustive list of prior art mentioned are typical of other marginally related prior art found in the process of a prior art searches. None of them were found specifically relating to the present invention since they do not disclose the use of shape wave in synergistic combination with magnetism for the purpose of eliminating or controlling the signs of baldness. The following patents reflect the state of the art, however, none of these patents teach or render obvious the claimed invention.

U.S. Pat. No. 5,827,170 to Gebran, entitled Magnetic Vortex, teaches a therapeutic device designed to be worn by a person. As distinguished from my invention, the magnetic flux of the prior art resides in the C or U-shaped magnet and a support plate is attached at both end regions of said magnet to hold a diamagnetic pyramid. Two opposing flanks of the pyramid face the N-pole (“N-pole”) and South pole (“S-pole”) of the magnet, thus causing the magnetic flux to avoid the diamagnetic pyramid and forming a varying distribution of lines of force around it. However, there is no indication nor is there any suggestion that Gebran considered applying his device to promote hair growth. Furthermore, Gebran's pyramid is of solid configuration designed to serve as an interceptor means, and is diamagnetic in composition in order to divert the magnetic flux. The Gebran patent teaches of a way of design ornamentation using combination magnetic health and body adornment or accessories, each having what appears to be positionally-fixed and stationary magnetic members.

JP60084139A2 to Naomichi, entitled Pyramid Energy Generating Apparatus, is developed by arranging a wire member and a composite body of the wire members along a conical surface and connected to a ring shaped upper support member and a ring shaped lower support member. Naomich teaches that when a pole of a magnet is contacted with a lower support member, a large amount of pyramid energy is directed to any direction. The apparatus is used for dehydration and corrosion prevention. There is no mention of its use for hair benefit nor for use as a massaging tool since there is no solid base to cover the underside of the apparatus but an open one.

JP10135031A2 to Hiroshi, entitled, Wavemotion Plate with Protrusions, comprises a magnetic material or steel plate on the surface of which numerous protrusions of quadrilateral pyramids are formed. The surface is painted gold and the back side is provided with an electromagnetic coil. According to Hiroshi, his wavemotion plate that induces current and voltage in living tissues “has the effect of reducing fatigue, curing lumbago, making the taste of cigarette and food such as beer milder, and increasing the combustion efficiency of an oil heater or the like.”

JP7250907A2 to Isamu, entitled, Magnetic Cushion Sheet, comprising projecting peak parts of a cushion material which are hardened and then formed a pyramidal shape. Permanent magnets are adhered below the hardened peak parts in the recessed part of the cushion mat. Again there is no mention of the patent's ability to address the hair loss problem, like the Hiroshi patents discussed above.

These related prior arts did not suggest nor consider the use of these devices to address the hair loss problem. The prior arts comprise devices and means substantially distinguishable from the present invention, There is no structural and functional means to position the magnets against scalp and skin for magnetic flux patterning to tap the body's own polarized centers. Additionally, they do not teach other means for varying polarity and magnetic differential, or positioning, or variance of magnetic component elements to achieve a particular therapeutic result.

Further, the configuration of the housing prevents the physical movement of the magnet within the interior of the housing. Magnets are placed within devices and are not moved over affected target tissues but are either secured to the user, like the Gebran patent, or are placed proximate the user, like the Naomichi, Hiroshi and Isamu patents.

Prior to the present invention, however, there has not been a device designed for home use which combines the use of pyramid energy, magnetic force and the circular effleurage with biological polarity patterning.

While various prior art devices apply constant or dynamic magnetic fields to various anatomical areas, none of the prior art device applies magnetic field through a geometrical structure and in conjunction with the prescribed massage and polarity positioning of the present invention.

Additionally, none of the related prior art devices of promoting hair growth provides the structural and functional ability to utilize positionable magnets to effect manual change of polarity; nor do they provide the ability to utilize such magnets in flexible and opposite poled embodiments to pass through the body's own magnetic circuit and make a complete magnetic path, to achieve greater flexible access and simultaneous magnetic effect of adjacent body parts and tissues.

Also, the prior art devices for hair regeneration do not address the circular sliding movements or, effleurage, for best affecting transmittal of energetic forces like the magnetic flux and the shape wave, nor do they have gripping means or part to manually effect said movement.

None of the prior art devices teaches and permit the permanent magnetic bodies to be particularly configured, patterned and arranged to meet particular therapeutic needs, such as the application of a magnetic field having substantially all N-pole flux or substantially all South Pole flux to access a half waveform, and also to be able to access a frill waveform energy treatment from the same device.

None of the prior art devices and method of promoting hair growth has provided an acceptable balance between cost and effectiveness, safety and effectiveness, cost and ease of use or application, or the most desirable which is the presence of all these important features. Accordingly, there is a need for such an inexpensive stand-alone device which is ergonomically adapted for repeated utilization by the user, and which is capable of utilization over a wide range of applications, besides hair regeneration.

These and other disadvantages, structurally and functionally, of the prior art, will become apparent in reviewing the remainder of the present specification, claims and drawings.

### BRIEF SUMMARY THE INVENTION

The present invention describes a bio-enhanced magnetic device that is simple in both is construction and use, and which to optimizes not only the stimulation of the scalp, or other tissue, but also the tissue absorption of magnetic energies by using, in combination, the harmonizing effect of the circular effleurage and the balancing effect of biological polarity patterning. The device is essentially a magnet situated in relation to a pyramid such that the magnetic flux emanating from the magnet is enhanced by the and shape wave generated by the pyramid. The S-pole surface is positioned to enable the delivery of a half waveform of positive magnetism to the scalp of the user, which has been deemed to be most effective in treatment of chronic affectations like the approaching signs of baldness.

The invention takes advantage of another aspect of the magnetic flux phenomenon. That other aspect is the presence of magnetic field not only around the earth and the cosmos, but also within the body of living organism where polarity centers and magnetic path flow form a continuous current flow in a closed circuit resembling a double helix or figure eight.

### DESCRIPTION OF THE DRAWINGS

The invention will be readily understood by reference to the following detailed description when considered in connection with the accompanying drawings in which:

FIG. 1 is a top perspective view of a first embodiment of the device of the invention.

FIG. 2 is a bottom perspective view of the device of FIG. 1.

FIG. 3 is a partly sectional cut-away view of the bottom portion of the device of FIG. 1.

FIG. 4 is a top perspective view of a second embodiment of the device of the invention.

FIG. 5 illustrates the helical/vortical movement of the shape wave in a pyramidal structure.

FIG. 6 illustrates the path of magnetic flux from the primary and secondary source of magnetism in the device of FIG. 4.

FIG. 7 depicts how the magnetic lines of flux penetrate into and operate on the human body.

FIG. 8 depicts how the half waveform from an S-pole and an N-pole outwardly of said poles to penetrate into human contacting parts.

FIG. 9 is a top perspective view of a third embodiment of the device of the invention.

FIG. 10 is a top perspective view of a fourth embodiment of the device of the invention having an open frame skeletal structure of three-sided pyramid.

FIG. 11 is a top perspective view of a fifth embodiment of the device of the invention having a generally conical magnetizable body, with the top portion in an exploded view.

### DETAILED DESCRIPTION OF THE INVENTION

Referring to the accompanying drawings wherein like reference numerals refer to the same or similar elements, in general, there is shown my invention for a bio-enhanced magnetic device for promoting hair growth, said magnetic device designated as 10.

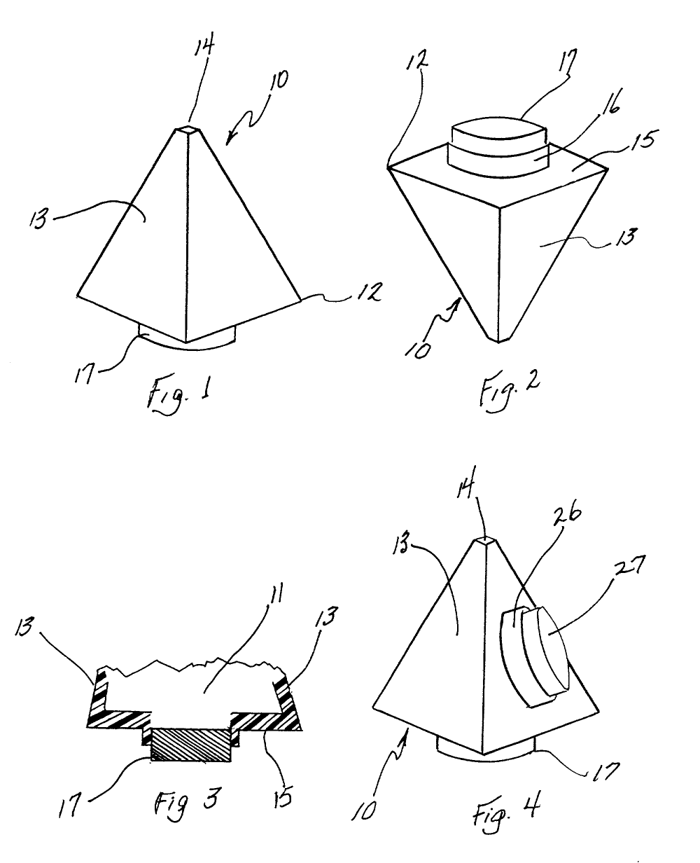

A first embodiment of the magnetic device of the invention is illustrated in FIGS. 1-3. The magnetic device 10 comprises a five-side hollow, generally pyramidal body 12, having a base 15, and four walls 13. Base 15 is of generally square shape, having formed at its geometric center a circular flange, or sleeve 16 defining a cylindrical cavity for receipt of a primary source of magnetism, permanent magnet 17. This cylindrical cavity may or may not provide an aperture to the interior of body 12. Each of the four walls 13 is triangularly shaped, with the interior of magnetic device 10 forming an amplifying chamber 11. The pyramidal body 12 is truncated at its apex 14. Sleeve 16 is sized to receive and snugly hold magnet 17. It should be understood that sleeve 16 need not necessarily be of cylindrical shape. Permanent magnets, as discussed below, come in various shapes and sizes, and sleeve 16 may be shaped and sized to receive a variety of magnets 17. FIG. 5 illustrates the vortical and helical movement of the shape wave due to the incline of the sides of the pyramid at a specific geometric configuration. The location of magnet 17 in base 15 places the magnetic lines of force emanating from the N-pole of the magnet in the vortical and helical field of chamber 11, thereby enhancing the quality and reach of the magnetic flux, and wherein which the magnetic flux density varies, and eddy currents are generated. In this way an enhanced unipolar magnetic field can be applied to the selected surface of the body. FIG. 3 shows magnet 17 with the N-pole surface defining a head section 19 facing the hollow interior of chamber 11 and the S-pole surface defining a base surface 18, for contact with the selected surface of the user. The S-pole portion is exposed in this case to deliver a half waveform of positive magnetism to the scalp of the user. Treatment of desired areas can be simply accomplished by effleuage of the desired area with the base surface 18 of device 10.

Magnetic members, which may either abut the chamber, be contained within the chamber, or come separately in separate magnet pieces, can also be of variety of shapes such as square, rectangular or oval, and can be made larger in diameter and thinner or thicker. By this means, one could, for example, double the magnetic field line density by doubling the number of magnets or magnet pieces, to be positioned in layer one over the other, whereby in each case N-pole of one magnet piece is opposite the S-pole of the adjacent magnet piece. This is configuration is particularly applicable when using low gauss rating or when a magnet piece larger in diameter is desired to cover larger areas of the body like the thigh and the buttocks. For treatment against falling hair, it has been found that the S-pole is more effective. Hence in various embodiments, the S-pole is positioned facing out from base 15 so that it will be in direct contact with the scalp, while the N-pole is to be found on the opposite section, or the head section 19, facing chamber 11. While permanent magnets of any strength may be utilized in the present invention, magnets having strengths within the range of 2,500 to 20,000 Gauss are preferred. The permanent magnets are preferably ceramic or rare earth magnets such as neodymium iron boron or samarium cobalt, although other magnetic bodies may be utilized. Any type of magnet is suitable for use herein in which the poles are sufficiently separated that the energy of each pole can be isolated from the energy of the other pole

The pyramidal body 12 of the amplifying chamber 11 is preferably made of hard but pliable and moldable plastic or other synthetic materials and preferably hollow inside for more efficient interaction of energies between the magnetic flux and the shape wave. In addition, the desired lightness of magnetic device 10 is maintained, though it could also be constructed in solid form especially if the natural-based materials, which also can be used, are chosen like the quartz crystal, semi-precious stones, rocks and other materials from the plant and mineral kingdom which are also known to emit energies of their own. The pyramidal body 12 of the chamber 11 is preferably sized in view of ergonomic considerations to provide a grippable device that is comfortable to hold in the hand of a typical intended user, to render the device efficient and non-tiring during use, as well as for safety considerations. To this end, the illustrated preferred embodiment of chamber 11 includes a truncated apex 14. The amplifying chamber 11 of the most preferred embodiment is also modeled in accordance with the geometrical configurations of the famous Great Pyramid at Gizah having a base to wall ratio of 7:11, to attain the goal of producing a varying field of magnetism that is necessary for optimization of magnetic energy.

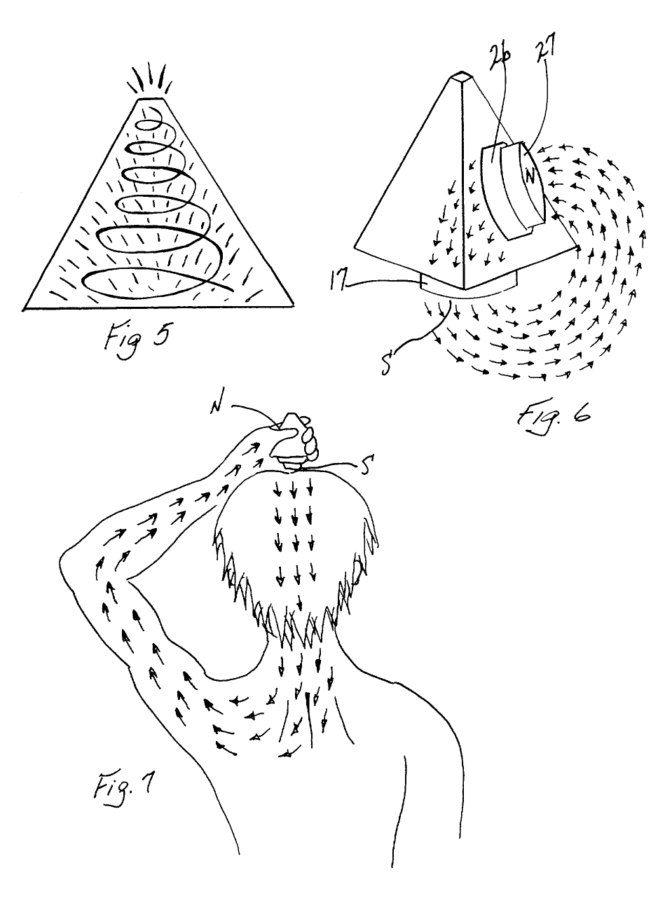

A second embodiment of the invention is shown in FIG. 4 wherein body 12 additionally includes the feature of one of the walls 13 having formed, at its geometric center, a circular flange, or sleeve 26 defining a cylindrical cavity for receipt of an auxiliary source of magnetism, permanent magnet 27. This auxiliary source of magnetism is provided to enable the body to receive more than one source of magnetism of reverse polarities at the same time. This is achieved by providing magnet 27 that can closely contact another body part of the user by disposing it about body 12 where the gripping hand contacts magnet 27 while applying the device 10 over the treatment area.

As shown, magnet 27 is positioned within sleeve 26. Magnet 27 has its N-pole surface defining base section 28 and its S-pole surface defining head section facing chamber 11. Thus the primary and auxiliary sources of magnetism are accordingly oriented so that base surfaces 18 and 28 of magnets 17 and 27 respectively., have opposite polarities. FIG. 6 depicts the path of magnetic field emanating from the two magnets. Since the magnets are positioned in close proximity to each other, there will be alternating zones of convergent and divergent flux (north and south) which will be strongest in the areas inside amplifying chamber 11. The area between the magnets will be filled with return path magnetic flux that will measure as opposite polarity on a Hall effect Gauss meter. This area called Bloch wall also contributes to the formation of varying magnetic field when acted upon by the shape wave.

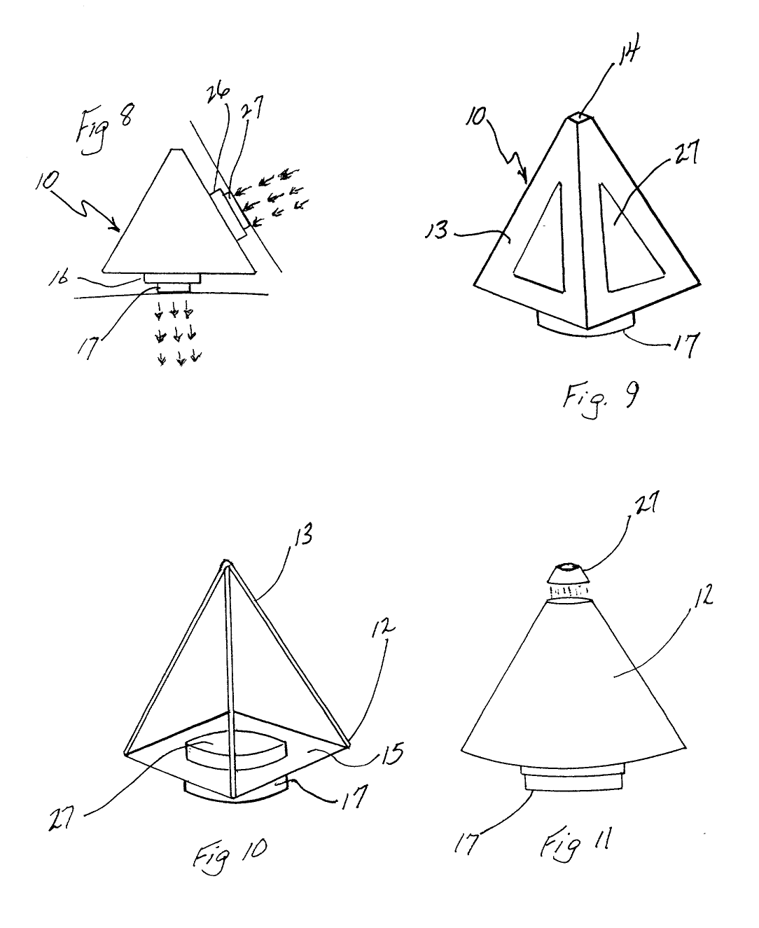

FIG. 7 illustrates the state in which the magnetic device of the embodiment in FIG. 4 is applied to the scalp. Magnetic fluxes from the S-pole to the N-pole detour, accessing the polarity of the gripping hand, to extend and read the opposite polarity at the end of the flux line, with the result that magnetic fluxes enter not only the surface of the tissues but also a deep portion of the body. Hence the benefit of full waveform magnetism, which in this case is the balancing of polarity energies occurring on the deeper part of the body, is likewise attained. FIG. 8 is a depiction of the magnetic flux emanating from one skin surface and returning to a second skin surface.

Auxiliary magnet 27 may also be disposed by other means than that depicted in the embodiment of FIG. 4. Auxiliary source of magnetism may be located within or without the chamber 11, as long as there is provided a magnet of opposing polarity that impinges the hand, or other body parts where a polarity center of similar polarity mode is present. Alternatively, the separate magnet may be in form of commercially available, flexible magnetic sheets which may be cut and styled to cover a greater portion of the hand or body parts. Additionally, as shown in FIG. 9, the commercially-sold flexible magnetic sheets may also be cut and affixed by means of gluing on the walls of the three sided or four-sided pyramidal embodiments or wrapped around and glued on a conical body, forming primary magnets 27, and the auxiliary magnet would then be magnet 17.

In the embodiment of open frame skeletal pyramid 12, as shown in FIG. 10, which may be constructed having three sides or four sides, auxiliary magnet 27 is located on top of the solid plate of base 15, attached therein by reason of its attracting force with permanent magnet 17, the N-pole of magnet 17 abutting the bottom of base 15 and the S-pole of magnet 27 abutting the upper surface of base 15. The N-pole of magnet 27 then faces the chamber 11. Magnets 17 and 27 may be removably attached on the solid plate of the base 15 where they can be interchanged or replaced or added with another layer of magnets. Base 15 can be made of materials different from the sides 13 to attain a certain purpose, such as from a solid magnetizable plate, eliminating the requirement of a sleeve to house magnet 17, but is easily attached, detached and replaced by reason of its attracting force. Alternately, the whole body, not only the base could be made of magnetizable material so that only one permanent magnetic member 17 is needed since by reason of its position at the base, the reverse polarity of said magnet will now be the polarity of the entire body of the device 10, providing the N-pole and the S-pole without having two magnets installed. However, when the magnets used are of substantially high Gauss rating, even when non-magnetizable materials are used, they can overcome the intervening non-magnetizable plate and still attract each other by reason of their high permeability.

A variety of geometric shapes and forms known to accumulate, direct, focus and transform energies, which include not only the pyramidal, but also conical and spherical forms, or a combination of these forms, may be employed in constructing magnetic device 10 to create an amplifying chamber 11. In the case of an alternative embodiment having a conical body 12 as shown in FIG. 11, auxiliary magnet 27 is disposed to sit on top of the substantially truncated portion of apex 14, said magnet 27 made of circular magnet with sloping sides and flat end surfaces. In other words, the shape of the magnet 17d is configured to keep the general conical shape and also the general truncation of the apex 14. The conical body 12 may be made of non-magnetizable material, in which case magnet 27 is attached on the top portion of the truncated apex by means of gluing, while the magnet 17 is disposed at the base by means of a sleeve 16. If magnetizable body is used, the magnets 17 and 27 will be attached to the body 12 by reason of their attracting force.

In the case where the structure is to be simplified, or if the number of parts is to be reduced, as in the case of the embodiment of FIG. 4, the magnet which is designed to provide auxiliary source of magnetism can come in form of separate or loose magnet piece or pieces that can be removably attached adjacent the palm or back of the hand with one face of the magnet always pointing toward the subject tissue. The separate magnets may also be in form of rectangular magnet which cover more areas compared to circular or square ones, or a magnet with a larger dimensions, or two or more magnets with the same or different sizes that are layered one over the other if greater coverage or greater magnetic flux is desired. They can be held in place by means of a tensioning wrap, or Velcro, or two-sided tapes, glue or other tacky substance or tensile fabric wraps.

The base section of the magnetic member may have a studded surface or a corrugated surface to effect efficient massaging application especially when the device is used in conjunction with a massaging cream or therapeutic oils. The magnetic member may be cladded with a fabric that could be impregnated with therapeutic or aromatherapeutic oils and cream.

As illustrated in FIG. 7, the device is held in such a way that one part of the body, for example the gripping hand, is exposed to a unipolar magnetic body, white a second part of the body, for example the afflicted scalp is also directly contacted by another unipolar magnetic body of opposite polarity, being lightly massaged therein. The light massage given to the afflicted area is a circular sliding massage, with very little force given like an effleurage, in a clockwise or counterclockwise direction. When the selected area is massaged by the device of the invention, a deeper area is reached compared with the prior arts magnetic pads and devices because the skin being in relaxed state offers no resistance to the penetration of the magnetic flux.

The single action of gripping the N-pole face of the auxiliary magnet 17 initially delivers a half waveform of negative mode of magnetism to the arm area. On the other hand, the next step of impinging the S-pole face of magnet 17 to an afflicted area of the body will result in a half waveform of positive mode of magnetism being delivered to said area.

In the context of the present invention, these newly appreciated properties of body magnetics are applied in a process herein called body magnetic patterning, such that the polarity of magnetic field is patterned after the corresponding polarity center of the living body so that an efficient and vitalized transfer of energy occurs, further energizing the body's own double-helix natural magnetic circuit. The N-pole magnet contacts the skin surface and the magnetic lines of force emitted from the N-pole penetrate into and operate on the human body, and thereafter reach the S-pole. Therefore, the magnetic lines of force produce a continuous magnetic flow path through the gripping hand and the sections of the body where the magnetic field completes its path, traveling along the polarity centers and then reading the opposite polarity at the other end of the line of force. The benefit of full waveform magnetism, which balances the energy, is thereby contemporaneously attained.

In the case of hair and scalp affliction including excessive falling hair and alopecia, the mode of magnetism needed by the afflicted area is the South or positive mode owing to the chronicity of the problem. To deliver the needed positive mode magnetism to the scalp, the South pole is applied to the scalp. See FIG. 7. Consequently according to the invention, an opposite mode, which in this case is the negative mode, should also directly contact another part of the body, and this is through the N-pole of the auxiliary magnet being gripped by a similarly polarized hand, which is the left hand bearing the inherent negative polarity center. This body magnetic patterning is made efficiently possible by the bio-enhanced magnetic device and at the same time it also doubles the therapeutic value of the device.

It is to be understood that while the description of the invention generally has referred to the treatment of falling hair and alopecia, it is contemplated that headache, sinusitis, arthritic pain, stiffness, and other acute and chronic symptoms of pain may also be beneficially treated by the device and method which are disclosed herein.

Having described specific embodiments of the invention with reference to the accompanying drawings, it is to be understood that the invention is not limited to those precise embodiments and that various changes and modifications may be effected therein by one skilled in the art without departing from the scope or spirit of the invention as defined in the appended claims.

I claim:

1. A portable, hand-held, bio-enhanced, magnetic device having a therapeutic value in promoting hair growth and alleviating pain in body parts of a living organism, the device comprising:
a specific geometrically-shaped body having sides and a base, the base having a top surface and a bottom surface, the base having a selected shape to complement the geometry of the sides, the base formed to have a means for receiving a primary magnet, the sides and the base defining a specific geometrically-shaped chamber therewithin, the chamber for amplifying a magnetic flux, the chamber adapted for generating shape wave energy;
the primary magnet of selected strength, having a north and south polarity for generating a magnetic flux, the magnet positioned such that the magnetic flux intersects the amplifying chamber; and
whereby when the primary magnet is inserted in the means for receiving a primary magnet, the amplifying chamber produces an enhanced magnetic flux emanating between the north polarity to the south polarity of the magnet, the device for directing the enhanced magnetic flux to the body of a user.

2. The device of claim 1 wherein the means for receiving a primary magnet comprises a sleeve, the sleeve extending from the bottom surface of the base, the size and shape of the sleeve selected to snugly receive the magnet about its periphery.

3. The device of claim 1 wherein said amplifying chamber is hollow.

4. The device of claim 1 wherein the primary magnet is selected to have a base surface of a selected polarity and a head surface of the opposite polarity, the magnet positioned such that the base surface is directed outwardly from the geometrically-shaped body, and the head surface is directed inwardly toward the amplifying chamber, the base surface for contact with the body of the user.

5. The device of claim 1 wherein the specific geometrically-shaped body additionally includes an auxiliary magnet in close proximity to the amplifying chamber.

6. The device of claim 5 wherein a selected side of the geometrically-shaped body additionally include a means for receiving the auxiliary magnet, the auxiliary magnet selected to have a base surface of a selected polarity and a head surface the opposite polarity, the magnet positioned such that the base surface is directed outwardly from the geometrically-shaped body, and the head surface is directed inwardly toward the amplifying chamber, the base surface for contact with the body of the user.

7. The device of claim 5 wherein the base surface of the primary magnet has a different polarity than the base surface of the auxiliary magnet, and
whereby more than one source of magnetism of reverse polarity are positioned to deliver a half waveform of magnetism of each polarity to two separate parts of the body of the user, and whereby the parts of the body of the user are subjected to a continuous and complete magnetic flux.

8. The device of claim 1 wherein said amplifying chamber is a generally pyramidal-shaped, having at least three sides and at least three walls, and with the base shaped to complement the at least three sides.

9. The device of claim 8 wherein the at least three sides are of an open skeletal frame structure and wherein the complementary-shaped base is solid and magnetizable and the amplifying chamber is hollow.

10. The device of claim 5 wherein the sides form a smooth, conical structure with a truncated apex, the truncated apex forming side of the geometrically-shaped body, and wherein:
the complementary-shaped base is solid and magnetizable;
the means for receiving the auxiliary magnet is the truncated apex;
the auxiliary magnet is shaped generally in the form of a truncated cone, and is sized and shaped to seat in the truncated apex of the conical structure; and
wherein the auxiliary magnet is removably secured to the apex of the conical structure through magnetic attraction of the primary magnet.

11. The device of claim 5 wherein the means for receiving the auxiliary magnet is disposed separately from the geometrically-shaped body, said means for receiving the auxiliary magnet positioned to place the auxiliary magnet in direct contact with a skin area of a gripping hand of the user.

12. A portable, hand-held, bio-enhanced, magnetic device having a therapeutic value in promoting hair growth and alleviating pain in body parts of a living organism, the device comprising:
a specific geometrically-shaped body having a base and sides, the base having a top surface and a bottom surface, the base having a selected shape and formed to have a means for receiving a primary magnet, the geometrically-shaped body defining a chamber therewithin, the chamber adapted for generating shape wave energy; the chamber for amplifying a magnetic force field;
at least one of the sides formed to have a means for receiving an auxiliary magnet;
the primary and auxiliary magnets of selected strength, having a north and south polarity for generating a magnetic force field, each magnet having a base surface and a head surface, the base surface for contacting with the body of a user, the head surface for communication with the amplifying chamber;
whereby when the primary magnet is inserted in the means for receiving the primary magnet such that the base surface has a selected polarity, and the auxiliary magnet is inserted in the means for receiving the auxiliary magnet such that the base surface of the auxiliary magnet has the opposite polarity, the magnets are positioned such that the magnetic force field of each magnet intersects the amplifying chamber, and the amplifying chamber produces an enhanced magnetic flux emanating between the primary magnet and the auxiliary magnet; and
wherein each the parts of the body of the user are subjected to an enhanced magnetic flux of half waveform of each polarity.

13. The device of claim 12 wherein said body defining the amplifying chamber is a generally pyramidal-shaped, having at least three sides and at least three walls, and with the base shaped to complement the at least three sides.

14. The bio-enhanced magnetic device of claim 13 wherein:
the at least three sides are of an open skeletal frame structure and a complementary shaped solid base made of magnetizable material;
the means for receiving the auxiliary magnet is the interior surface of the base; and
whereby the primary and auxiliary magnets are selectively and interchangeably positionable by reason of their attracting force to enable the user to select the magnetic flux polarity that will directly contact the body parts of the user.

15. The device of claim 12 wherein the sides form a smooth, conical structure with a truncated apex, and wherein:
the complementary shaped base is solid and magnetizable;
the means for receiving the auxiliary magnet is the truncated apex;
the auxiliary magnet is shaped generally in the form of a truncated cone, and is sized and shaped to seat in the truncated apex of the conical structure; and
wherein the auxiliary magnet is removably secured to the apex of the conical structure through magnetic attraction of the primary magnet.

16. A method for promoting hair growth and alleviating pain in body parts of a living organism, the method comprising:
subjecting a specific geometrically-shaped body having a chamber adapted for generating shape wave energy to a magnetic flux, amplifying a magnetic flux in the chamber; and
directing the enhanced magnetic flux to the body of a user.

17. The method of claim 16 wherein the step of subjecting a specific geometrically-shaped body to a magnetic flux includes the step of placing a primary magnet in close proximity to the chamber and contacting the user with the primary magnet.

18. The method of claim 16 wherein the step of subjecting a specific geometrically-shaped body to a magnetic flux includes the step of placing a secondary magnet in close proximity to the chamber and contacting the user with the auxiliary magnet.

19. The method of claim 16 wherein the geometrically-shaped body is of symmetrical, pyramidal structure.

20. The method of claim 19 wherein the symmetrical, pyramidal structure is of skeletal form, and the step of placing the secondary magnet in close proximity to the chamber includes the step of placing the auxiliary magnet adjacent the primary magnet.

21. The method of claim 19 wherein the symmetrical, pyramidal structure is of smooth, conical shape with a truncated apex, and the step of placing the secondary magnet in close proximity to the chamber includes the step of placing the auxiliary magnet at the truncated apex.
